(19) United States
(12) Patent Application Publication
(10) Pub. No.: US 2024/0165235 A1
(43) Pub. Date: May 23, 2024

(54) ULTRASMALL GADOLINIUM-INTERCALATED CARBON DOTS AS A RADIOSENSITIZING AGENT FOR CANCER

(71) Applicant: UNIVERSITY OF GEORGIA RESEARCH FOUNDATION, INC., Athens, GA (US)

(72) Inventor: Jin XIE, Athens, GA (US)

(21) Appl. No.: 18/282,705

(22) PCT Filed: Mar. 18, 2022
(86) PCT No.: PCT/US2022/020862

§ 371 Date: Sep. 18, 2023

Related U.S. Application Data
(60) Provisional application No. 63/163,020, filed on Mar. 18, 2021.

(51) Int. Cl. A61K 41/00; A61K 9/51; A61K 33/244; A61K 33/44; A61P 35/00

(52) U.S. Cl. CPC A61K 41/0038; A61K 9/5115; A61K 33/244; A61K 33/44; A61P 35/00

(57) ABSTRACT

Gd-encapsulated carbonaceous dots (Gd@C-dots) hold great potential in clinical translation as T₁ contrast agent for magnetic resonance imaging. Disclosed are methods of using said particles as radiosensitizing agents.

### ACKNOWLEDGEMENT OF GOVERNMENT SUPPORT

This invention was made with government support under Grant No. EB022596 and CA247769 awarded by the National Institutes of Health. The government has certain rights in this invention.

### BACKGROUND

High-Z nanoparticles (HZNPs) are an emerging type of radiosensitizer. HZNPs can increase the production of photo- and Auger-electrons under high energy beams, thus enhancing the efficacy of radiotherapy (RT). Several groups reported that gold nanoparticles can augment cellular damage under KV and MV beams. Hafnium oxide nanoparticles (NBTXR3) and Gd-chelate-bound silica nanoparticles (AGuIX) have been tested in the clinic; the former were approved in the Europe for treatment of locally advanced soft tissue sarcoma. Bi-, Pt-, and W-containing HZNPs have also been synthesized and investigated. Despite the promise, however, toxicity remains a major concern for HZNPs. Many HZNPs are often made of metals or metal oxides that may degrade over time to release toxic heavy metals. HZNPs made from inert materials cause less acute toxicity but may stay months in the host, and their long-term impact remains to be fully investigated. Furthermore, many conventional HZNPs have relatively large sizes (e.g. 20-200 nm in diameter), which limit their accumulation in tumors and uptake by cancer cells. Due to these restrictions, HZNPs are often injected intratumorally rather than systemically, which may limit their potential applications in the clinic. The subject matter disclosed herein addresses these and other needs.

### SUMMARY

In accordance with the purposes of the described materials, compounds, compositions, articles, and methods, as embodied and broadly described herein, the subject matter described herein, in one aspect, relates to compositions and methods for preparing and using such compositions.

In one aspect, disclosed herein are methods of making a subject with a cancer (such as, for example, non-small cell lung cancer) more susceptible to radiotherapy comprising administering to the subject gadolinium-intercalated carbon dots.

Also disclosed herein are methods of increasing the efficacy of radiation treatment to a cancer (such as, for example, non-small cell lung cancer) comprising administering gadolinium-intercalated carbon dots to a subject receiving radiotherapy for the treatment of a cancer. In one aspect, the increased efficacy can occur due to an increase in hydroxyl radical production. Accordingly, disclosed herein are methods of increasing hydroxyl radical production in a tumor in subject undergoing x-ray irradiation comprising administering to the subject gadolinium-intercalated carbon dots and radiotherapy.

In one aspect, disclosed herein are methods of treating, inhibiting, reducing, decreasing, ameliorating, and/or preventing a cancer and/or metastasis (such as, for example, non-small cell lung cancer) in a subject with a cancer comprising administering to the subject gadolinium-intercalated carbon dots and radiotherapy.

Also disclosed are methods of making a subject more susceptible to radiotherapy of any preceding aspect; methods of increasing the efficacy of radiation treatment of any preceding aspect; methods of increasing hydroxyl radical production in a tumor of any preceding aspect, methods of treating, inhibiting, reducing, decreasing, ameliorating, and/or preventing a cancer and/or metastasis of any preceding aspect; wherein the gadolinium-intercalated carbon dots further comprise a carboxylic acid or amino group and/or a detectable label.

Also disclosed herein are making a subject with a cancer (such as, for example, non-small cell lung cancer) more susceptible to radiotherapy of any preceding aspect, methods of increasing the efficacy of radiation treatment to a cancer (such as, for example, non-small cell lung cancer) of any preceding aspect, and/or methods of treating, inhibiting, reducing, decreasing, ameliorating, and/or preventing a cancer and/or metastasis (such as, for example, non-small cell lung cancer) of any preceding aspect, wherein the gadolinium-intercalated carbon dots are administered concurrently with the administration of radiotherapy or administration begins 1, 2, 3, 4, 5, 10, 15, 20, 25, 30, 35, 40, 45, 50, 55, 60, 70, 75, 90, 105, 120 minutes, 3, 4, 5, 6, 7, 8, 9, 10, 11, 12, 13, 14, 15, 16, 17, 18, 19, 20, 21, 22, 23, 24, 30, 36, 42, 48, 54, 60, 66.72 hours, 4, 5, 6, 7, 8, 9, 10, 11, 12, 13, 14, 15, 16, 17, 18, 19, 20, 21, 22, 23, 24, 25, 26, 27, 28, 29, 30, 45, 58, 59, 60, 62, 62, or 90 before the administration of radiotherapy. In some instances administration of the gadolinium-intercalated carbon dots can begin 1, 2, 3, 4, 5, 10, 15, 20, 25, 30, 35, 40, 45, 50, 55, 60, 70, 75, 90, 105, 120 minutes, 3, 4, 5, 6, 7, 8, 9, 10, 11, 12, 13, 14, 15, 16, 17, 18, 19, 20, 21, 22, 23, 24, 30, 36, 42, 48, 54, 60, 66.72 hours, 4, 5, 6, 7, 8, 9, 10, 11, 12, 13, 14, 15, 16, 17, 18, 19, 20, 21, 22, 23, 24, 25, 26, 27, 28, 29, 30, 45, 58, 59, 60, 62, 62, or 90 after the administration of radiotherapy.

In one aspect, disclosed herein are making a subject with a cancer (such as, for example, non-small cell lung cancer) more susceptible to radiotherapy of any preceding aspect, methods of increasing the efficacy of radiation treatment to a cancer (such as, for example, non-small cell lung cancer) of any preceding aspect, and/or methods of treating, inhibiting, reducing, decreasing, ameliorating, and/or preventing a cancer and/or metastasis (such as, for example, non-small cell lung cancer) of any preceding aspect, wherein the gadolinium-intercalated carbon dots comprise nanoparticles of gadolinium encapsulated in an amorphous shell and a mesoporous silica nanoparticle. In one aspect, the mesoporous silica nanoparticle has an average diameter of from about 100 nm to about 200 nm. In some aspects, the mesoporous silica nanoparticle has an average pore size of from about 1 nm to about 20 nm, preferably, the mesoporous silica nanoparticle has an average pore size of about 3, about 7, or about 11 nm. In some aspects, the nanoparticles of gadolinium have an average diameter of from about 1 nm to about 20 nm.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DECRIPTION OF THE FIGURES

The accompanying figures, which are incorporated in and constitute a part of this specification, illustrate several aspects of the disclosure, and together with the description, serve to explain the principles of the disclosure.

FIGS. 1A, 1B, 1C, 1D, and 1E show the size and composition analysis of Gd@Cdots. FIG. 1a shows a representative TEM image of Gd@Cdots. The average particle size was 2.60 nm. FIG. 1B shows a hydrodynamic size of Gd@Cdots, measured by DLS. FIG. 1C shows a zeta potential analysis of Gd@Cdots. The nanoparticles carry a positive surface charge (+33.3 mV). FIG. 1D shows elemental analysis by EDS. Molar ratios between carbon and nitrogen or gadolinium were presented. FIG. 1E shows mass spectra of Gd@Cdots, analyzed using LDI-TOF (left) and ESI (right).

FIGS. 2A, 2B, 2C, 2D, 2E, and 2F show physical characterizations of Gd@Cdots. FIG. 2A shows Gd³⁺ released from Gd@Cdots in neutral and acidic solutions (pH 7.0 and 5.0) at 37° C. for 24 h, evaluated by ICP-MS. FIG. 2B shows absorbance spectrum of Gd@Cdots. There peaks at 457, 520, and 570 nm were observed. FIG. 2C shows fluorescence spectra of Gd@Cdots. Emission peaks around 630 nm were observed when the nanoparticles were excited by 457, 520, and 570 nm light. FIG. 2D shows fluorescence intensity when Gd@Cdots were incubated in 10% FBS or 1 mM GSH for 24 h at 37° C. Compared to pre-incubation solutions, minimal fluorescence change was observed over the incubation, indicating high stability of the nanoparticles. FIGS. 2E and 2F show phantom studies with Gd@Cdots agarose gel samples (Gd concentration 0.00018-0.094 mM), measured on a 7 T magnet. FIG. 2E shows T₁ and T₂ MR images of Gd@Cdots gel samples. FIG. 2F shows r₁ and r₂ relaxation of Gd@Cdots, evaluated based on results from e).

FIGS. 3A, 3B, 3C, and 3D show radical production in the presence of Gd@Cdots (30 μg/mL). FIG. 3A shows ROS level changes, evaluated by measuring methylene blue fluorescence change. *p<0.05. FIG. 3B shows singlet oxygen (¹O₂) production, measured by SOSG assay. *p<0.05. FIG. 3C shows hydroxyl radical (·OH) generation, measured using TA as a probe. *p<0.05. FIG. 3D shows hydroxyl radical production in the presence of Triton X-100.

FIGS. 4A and 4B. Cell uptake of Gd@Cdots. FIG. 4A shows fluorescence microscopy analysis. LysoTracker and MitoTracker green were as counter-stains to label endosomes/lysosomes and mitochondria, respectively. FIG. 4B shows STEM analysis of sectioned cell samples. Clusters of Gd@Cdots in the endosomes were visualized.

FIGS. 5A, 5B, 5C, 5D, and 5E show the impact of Gd@Cdots on cell viability, evaluated with H1299 cells. FIG. 5A shows cell viability in the absence of radiation, measured by MTT assay. FIG. 5B shows cell viability under radiation (5 Gy), measured by ATP bioluminescence assay. Gd@Cdots (30 μg/mL) were incubated with cells during radiation. Cdots were also tested as a comparison. FIG. 5C shows mitochondrial membrane potential change, measured by JC-1 staining. FIG. 5D shows cytochrome c release, evaluated by cytochrome c and mitochondria double staining. Cytochrome c translocation into the cytosol was indicated by red arrows. FIG. 5e shows activation of apoptosis, evaluated by caspase 3 activity assay. *, p<0.05.

FIGS. 6A, 6B, 6C, and 6D show radiosensitizing effects, tested in H1299 cells with Gd@Cdots (30 μg/mL). FIG. 6A shows lipid peroxidation, measured by Image-iT BODIPY assay. A decrease of 590/510 nm fluorescence ratio indicates an increased level of lipid peroxidation. *, p<0.05. FIG. 6B shows DNA damage, measured by rH2AX staining. The images were acquired on an ImageStream X Mark II Imaging Flow Cytometer. FIG. 6C shows clonogenic assay results. H1299 cells treated with Gd@Cdots (30 μg/mL) or PBS only and received 0-10 Gy radiation. The results were fit into an linear-quadratic equation: S(D)/S(0)=exp-(aD+bD²), where S is cell Survival fraction, D is radiation dose, and a&b are coefficients. FIG. 6D shows radiation enhancement factor at 4 Gy (REF4), computed based on results from 6C.

FIGS. 7A, 7B, 7C, 7D, and 7E show In vivo therapy results, tested in a H1299 bearing xenograft model. The animals were treated with Gd@Cdots (0.1 mmol Gd/kg) plus 6 Gy radiation (Gd@Cdots+RT), radiation only (RT), or PBS only (n=5). FIG. 6A shows tumor growth curves. *, p<0.05. FIG. 6B shows Kaplan Meir survival curves. FIG. 6C shows histology analysis of tumor samples. Both H&E and TUNEL assays were performed. Scale bars, 500 μm. FIG. 6D shows body weight curves. No significant body weight drop was observed throughout the studies. FIG. 6E shows histology analysis of major organ samples by H&E staining. Scale bars, 200 μm.

FIG. 8 shows As-synthesized Gd@Cdots dispersed in water.

FIG. 9 shows a STEM image of Gd@Cdots.

FIG. 10 shows the statics on size the distribution of Gd@Cdots, based on TEM results.

FIG. 11 shows Gd release when Gd@Cdots were incubated in 10% fetal bovine serum (FBS) or solutions containing 1 mM glutathione (GSH). Gd was quantified by ICP-MS.

FIG. 12 shows Gd@Cdots accumulation in mitochondria (stained with MitoTracker green). Images were acquired on an ImageStream X Mark II Imaging Flow Cytometer.

FIG. 13 shows statistics for cell γH2AX loci numbers, evaluated by ImageStream X Mark II Imaging Flow Cytometer.

FIG. 14 shows clonogenic assay with Au nanoparticles (10 μg/mL) and linear-quadratic data fitting.

FIGS. 15A and 15B show in vivo MRI scans. The studies were performed with H1299 tumor bearing nude mice (n=3) on a Varian 7T magnet. When the tumor size reached 500 mm₃, Gd@Cdots (0.1 mmol/kg based on Gd) were intravenously injected. Transverse and coronal T₁-weighted MR images were obtained at 0.5, 1, 2, 4, 6, and 72 h using the following parameters: field-of-view (FOV)=70×70 mm₂, TR/TE=500/12 ms, matrix size=256×256, slice=4, thickness=1 mm. The signal to background ratio was calculated using ImageJ software. FIG. 15A shows T₁-weighted MRI images, taken at different time points after Gd@Cdots injection (0.1 mmol Gd/kg). FIG. 15B shows signal-to-background ratios (SNRs), based on imaging results from 15A.

FIG. 16 is a cartoon showing a summary of the benefits of the Gd@Cdots for radiotherapy.

### DETAILED DESCRIPTION

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Methods of Using Gd@Cdots as Radiosensitizing Agents for the Treatment of Cancer

High-Z nanoparticles (HZNPs) are an emerging type of radiosensitizer. HZNPs can increase the production of photo- and Auger-electrons under high energy beams, thus enhancing the efficacy of radiotherapy (RT). Several groups reported that gold nanoparticles can augment cellular damage under KV and MV beams. Hafnium oxide nanoparticles (NBTXR3) and Gd-chelate-bound silica nanoparticles (AGuIX) have been tested in the clinic; the former were approved in the Europe for treatment of locally advanced soft tissue sarcoma. Bi-, Pt-, and W-containing HZNPs have also been synthesized and investigated. Despite the promise, however, toxicity remains a major concern for HZNPs. Many HZNPs are often made of metals or metal oxides that may degrade over time to release toxic heavy metals. HZNPs made from inert materials cause less acute toxicity but may stay months in the host, and their long-term impact remains to be fully investigated. Furthermore, many conventional HZNPs have relatively large sizes (e.g. 20-200 nm in diameter), which limit their accumulation in tumors and uptake by cancer cells. Due to these restrictions, HZNPs are often injected intratumorally rather than systemically, which may limit their potential applications in the clinic.

Herein we explored ultrasmall Gd-encapsulated carbon dots, or Gd@Cdots, as a radiosensitizing agent. We have synthesized Gd@Cdots and shown their potential as a magnetic resonance imaging (MRI) contrast agent as well as showing that Gd@Cdots can accumulate in tumors through the enhanced permeability and retention (EPR) effect. We postulate that intravenously (i.v.) administered Gd@Cdots can enhance energy deposition in tumors and improve RT tumor management. Because Gd@Cdots show minimal Gd leakage (due to a biologically and chemically inert carbon coating) and efficient renal clearance (due to ultrasmall particle sizes), we also expect the treatment to cause minimal side effects. We tested these hypotheses first in vitro and then in vivo in mice bearing H1299 tumors, which originated from human non-small cell lung cancer (NSCLC). NSCLC is diagnosed in more than 187,000 persons each year in the US and is a leading cause of cancer-related mortality. RT is the standard care for the majority of NSCLC patients with locally advanced (T3-4) or local regional disease (N2-N3), which accounts for ˜50% of newly diagnosed NSCLC cases. Despite technological advances, the rates of local failure in stage III NSCLC have remained high. Increasing radiation doses does not improve survival and may be harmful. Hence, there is an unmet clinical need for efficient and safe radiosensitizers for radiotherapy against NSCLC.

A number of studies have demonstrated radiosensitizing effects with gold, hafnium oxide, and gadolinium nanoparticles under KV and MV beams. However, HZNPs are often associated with issues such as slow clearance and heavy metal toxicity. Unlike conventional HZNPs that are often made of a single-component metal or metal oxide, Gd@Cdots are a composite nanomaterial. Heavy metals, in this case Gd, are tightly intercalated into a carbon matrix; the latter serves as a low-toxic and biologically inert capsule that effectively prevents heavy metal leakage and toxicity. Meanwhile, carbon is electronically active, helping dissipate photoelectrons from high-Z centers to the surroundings. The surface catalytic effects of carbon also contribute to radical production under radiation. It is also worth mentioning that Gd@Cdots afford strong fluorescence and magnetic properties that allow them to be traced both macroscopically and microscopically. Indeed, our studies show that Gd@Cdots accumulation in tumors can be monitored by MRI (FIG. S8); this property may allow for image-guided radiation that improves radiation delivery accuracy. Overall, the composite nature is linked to a number of merits that improve the biocompatibility and radiosensitizing effects of Gd@Cdots. Moreover, because the photoelectric effects and surface effects depend on two components of the particle, each of which can be adjusted, the composition of the particles can be tuned to further enhance the radiosensitizing effects. For instance, other metals, including heavier elements such as Eu and Bi, can be encapsulated into the carbon shell. It is also feasible to change carbon precursors, so that Gd@Cdots of different shell compositions and surface properties can be yielded; these changes may in turn affect the cellular uptake, biodistribution, and radiosensitizing effects of Gd@Cdots.

Accordingly, in one aspect, disclosed herein are methods of making a subject with a cancer (such as, for example, non-small cell lung cancer) more susceptible to radiotherapy comprising administering to the subject gadolinium-intercalated carbon dots. As a result of the gadolinium-intercalated carbon dots administration, the amount of radiation administered to a subject can be decreased. It is understood and herein contemplated that by making the subject more radiosensitive, not only can the dosage of the radiation administered by lowered, but the efficacy of the treatment increases. Therefore, also disclosed herein are methods of increasing the efficacy of radiation treatment to a cancer (such as, for example, non-small cell lung cancer) comprising administering gadolinium-intercalated carbon dots to a subject receiving radiotherapy for the treatment of a cancer. A further benefit of the use of gadolinium-intercalated carbon dots is that in conjunction with x-ray radiation rumors contacted with r gadolinium-intercalated carbon dots have increased hydroxyl radical production. Thus, in one aspect, disclosed herein are methods of increasing hydroxyl radical production in a tumor in subject undergoing x-ray irradiation comprising administering to the subject gadolinium-intercalated carbon dots and radiotherapy.

It is understood and herein contemplated that increasing the sensitivity of a subject to radiotherapy, the efficacy of radiotherapy, and the amount of hydroxyl radical production has a therapeutic benefit to a subject with a cancer. Thus, gadolinium-intercalated carbon dots to can be used in the treatment, reduction, decrease, inhibition, amelioration and/or prevention of a cancer and/or metastasis (such as, for example, non-small cell lung cancer). Accordingly, one aspect, disclosed herein are methods of treating, inhibiting, reducing, decreasing, ameliorating, and/or preventing a cancer and/or metastasis (such as, for example, non-small cell lung cancer) in a subject with a cancer comprising administering to the subject gadolinium-intercalated carbon dots and radiotherapy.

The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

In one aspect, it is understood the treatment of cancer does not need to be limited to the administration of Gd@Cdots alone or with radiaotherapy, but can include the further administration of anti-cancer agents to treat, inhibit, reduce, decrease, ameliorate, and/or prevent a cancer or metastasis. Anti-cancer therapeutic agents (such as chemotherapeutics, immunotoxins, peptides, and antibodies) that can be used in the methods of treating, inhibiting, reducing, decreasing, ameliorating, and/or preventing a cancer and/or metastasis and in combination with any of the disclosed neoantigens or any CAR T cells, TIL, or MIL specific for said neoantigen can comprise any anti-cancer therapeutic agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

In one aspect, disclosed herein are making a subject with a cancer (such as, for example, non-small cell lung cancer) more susceptible to radiotherapy, methods of increasing the efficacy of radiation treatment to a cancer (such as, for example, non-small cell lung cancer), methods of increasing hydroxyl radical production in a tumor, and/or methods of treating, inhibiting, reducing, decreasing, ameliorating, and/or preventing a cancer and/or metastasis (such as, for example, non-small cell lung cancer), wherein the gadolinium-intercalated carbon dots are administered concurrently with the administration of radiotherapy or administration begins 1, 2, 3, 4, 5, 10, 15, 20, 25, 30, 35, 40, 45, 50, 55, 60, 70, 75, 90, 105, 120 minutes, 3, 4, 5, 6, 7, 8, 9, 10, 11, 12, 13, 14, 15, 16, 17, 18, 19, 20, 21, 22, 23, 24, 30, 36, 42, 48, 54, 60, 66.72 hours, 4, 5, 6, 7, 8, 9, 10, 11, 12, 13, 14, 15, 16, 17, 18, 19, 20, 21, 22, 23, 24, 25, 26, 27, 28, 29, 30, 45, 58, 59, 60, 62, 62, or 90 before the administration of radiotherapy. In some instances administration of the gadolinium-intercalated carbon dots can begin 1, 2, 3, 4, 5, 10, 15, 20, 25, 30, 35, 40, 45, 50, 55, 60, 70, 75, 90, 105, 120 minutes, 3, 4, 5, 6, 7, 8, 9, 10, 11, 12, 13, 14, 15, 16, 17, 18, 19, 20, 21, 22, 23, 24, 30, 36, 42, 48, 54, 60, 66.72 hours, 4, 5, 6, 7, 8, 9, 10, 11, 12, 13, 14, 15, 16, 17, 18, 19, 20, 21, 22, 23, 24, 25, 26, 27, 28, 29, 30, 45, 58, 59, 60, 62, 62, or 90 after the administration of radiotherapy.

Also disclosed herein are making a subject with a cancer (such as, for example, non-small cell lung cancer) more susceptible to radiotherapy, methods of increasing the efficacy of radiation treatment to a cancer (such as, for example, non-small cell lung cancer), methods of increasing hydroxyl radical production in a tumor, and/or methods of treating, inhibiting, reducing, decreasing, ameliorating, and/or preventing a cancer and/or metastasis (such as, for example, non-small cell lung cancer), wherein the gadolinium-intercalated carbon dots comprise nanoparticles of gadolinium encapsulated in an amorphous shell and a mesoporous silica nanoparticle. In one aspect, the mesoporous silica nanoparticle has an average diameter of from about 100 nm to about 200 nm. In some aspects, the mesoporous silica nanoparticle has an average pore size of from about 1 nm to about 20 nm, preferably, the mesoporous silica nanoparticle has an average pore size of about 3, about 7, or about 11 nm. In some aspects, the nanoparticles of gadolinium have an average diameter of from about 1 nm to about 20 nm.

In one aspect, the gadolinium-intercalated carbon dots used any of the methods of making a subject more susceptible to radiotherapy disclosed herein; methods of increasing the efficacy of radiation treatment disclosed herein; methods of increasing hydroxyl radical production in a tumor disclosed herein, and/or methods of treating, inhibiting, reducing, decreasing, ameliorating, and/or preventing a cancer and/or metastasis disclosed herein can further comprise a label. As used herein, a label can include a fluorescent dye, a member of a binding pair, such as biotin/streptavidin, a metal (e.g., gold or copper), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorimetric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson—; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DUPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DilC18(3)); I Dinitrophenol; DiO (DiOC18(3)); DiR; DiR (DilC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two-step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

It is further understood and herein contemplated that the gadolinium-intercalated carbon dots used any of the methods of making a subject more susceptible to radiotherapy disclosed herein; methods of increasing the efficacy of radiation treatment disclosed herein; methods of increasing hydroxyl radical production in a tumor disclosed herein, and/or methods of treating, inhibiting, reducing, decreasing, ameliorating, and/or preventing a cancer and/or metastasis disclosed herein can further comprise a carboxylic acid or amino group.

### Methods of Making Gadolinium-Intercalated Carbon Dots

C-dots hold great potential in a wide range of applications such as in vivo imaging, intraoperative imaging, and immunofluorescence staining (Lim, S., et al., Chem. Soc. Rev. 2015, 44:362). A myriad of C-dot synthesis methods have been developed, including laser ablation, electrochemical oxidation, chemical oxidation, thermal carbonization, pyrolysis, and microwave irradiation (Wang, Y., et al., J. Mater. Chem. C 2014, 2:6921). Despite the diversity in synthesis strategies, however, it is generally challenging to control the size of C-dots. This is because the raw products from conventional syntheses often contain carbon species of varied sizes and architectures (Id.). Multiple rounds of washing and purification are often required to obtain particles of the desired qualities, leading to time-consuming preparation and low production yields (Miao, P., et al., Nanoscale 2015, 7:1586). Moreover, as-synthesized C-dots often show weak luminescence, and require a post-synthesis surface modification to “enlighten” the particles (Yang, S., et al., J. Am. Chem. Soc. 2009, 131:11308). The surface modification adds to the complications of quality control (Sun, Y., et al., J. Am. Chem. Soc. 2006, 128:7756).

Disclosed herein is a MSN-templated synthesis method for the preparation of gadolinium carbon dots. MSNs of different pore sizes were prepared using cetyl trimethylammonium bromide (CTAB)-templated co-condensation method, followed by alkaline-etching (Chen, Y., et al., Small 2011, 7:2935). The MSNs can be used as reactors, into which Gd precursors are loaded. The resulting conjugates can then be calcined, leading to formation of Gd@C-dots throughout the silica matrix. Restrained by the dimension of the pores, however, the nanoparticle growth is limited, yielding homogeneous products whose sizes mold by the silica pores. Taking MSN-3, -7, and -11 for instance (3, 8, 10 indicate the average pore size of the MSNs), the resulting Gd@C-dots are on average 3.0, 7.4, and 9.6 nm in size, respectively. The resulting nanoparticles show excellent magnetic and optical properties. In particular, 3.0 nm Gd@C-dots possess very high relaxivity of 10 mM⁻¹s⁻¹ and quantum yield of 30.2%. When coupled with a tumor targeting ligand, c(RGDyK), the resulting conjugates show great tumor targeting, with the unbound particles quickly cleared from the host. The synthetic method addresses one critical problem in synthesis of carbon nanoparticle, promising clinical translation of Gd@C-dots as a new type of imaging agents. The disclosed method permits preparation of homogenous particles without any size enrichment steps. More surprisingly, the resulting Gd@C-dots show excellent luminescence properties without further surface modification.

Including organofunctional silanes like APS in the MSN preparation has proven to be helpful for Gd@C-dot generation: when using MSNs made from pure TEOS as reactors, the same calcination protocol failed to produce Gd@C-dots. It is postulated that by using aminosilanes in coagulation, many primary and secondary amine groups are introduced into the silica matrix. These amines may loosely complex with Gd precursors that are loaded into the MSNs. More importantly, the amines serve as defacto defects in the silica matrix, which are oxidized during calcination. The oxidization is likely facilitated by metallic centers, causing carbonization and the growth of a carbon shell surrounding Gd. The growth, however, is limited by the volume of the silica cavity, leading to formation of Gd@C-dots of similar sizes.

The size effect on the r₁ relaxivity of Gd@C-dots is to some degree unexpected. For conventional Gd complexes or nanoparticles, increasing the size of the agents would result in an increased rotation time, leading to r₁ increase (Wang, Y., et al., Adv. Mater. 2015, 27:3841). For Gd@C-dots, however, size is inversely correlated to r₁. In fact, it is counterintuitive that Gd encapsulated carbon species have a high r₁. According to classic models, direct Gd-water interaction is needed for T₁ shortening, which, in our case, is not happening when Gd is encased within a layer of carbon. Yet, enhanced r₁ relaxivities are observed in different types of Gd encapsulated carbon species, including not only the disclosed Gd@C-dots, but also Gd loaded carbon tubes and fullerene (Holt, B., et al., ACS Appl. Mater. Interfaces, 2015, 7:14593). A possible explanation was given by Wilson, who proposed that the encased Gd cations may affect the electron density of the carbon shell, thus enabling the relaxation of water molecules to occur at the carbon shell without direct Gd-water interaction (Shu, C., et al., Biocon. Chem. 2009, 20:1186). It is also possible that the large number of hydroxide and carbonyl groups on the carbon shell play a role by facilitating the proton exchange of protons with the surroundings (Bolskar, R., et al., J. Am. Chem. Soc. 2003, 125:5471; Laus, S., et al., J. Phys. Chem. C 2007, 111:5633; Sitharaman, B., et al., Chem. Commun. 2005, 3915; d) Sithararnan, B., et al., Int. J. Nanomed. 2006, 1:291). With a reduced particle size, the ratio of surface or near-surface Gd contributing to the T₁ shortening is increased, thus leading to an enhanced r₁.

The luminescence of C-dots is also an interesting phenomenon and the mechanism has been a subject of debate. However, it is increasingly accepted that the radiative recombinations of the surface-confined electrons and holes are responsible for the luminescence (Zhu, S., et al., Adv. Funct. Mater. 2012, 22:4732). Previously, the most intensively studied factor for luminescence was surface passivation (Luo, P., et al., J. Mater. Chem. B 2013, 1:2116). It was found that proper post-synthesis surface modification, such as oxidation or tethering molecules to the particles surface, could dramatically enhance the luminescence intensity of C-dots (Liu, F., et al., Adv. Mater. 2013, 25:3657). In fact, post-synthesis surface modification is often times an essential step in preparation of luminescent C-dots (Yang, S., et al., J. Am. Chem. Soc. 2009, 131:11308). In the disclosed methods, however, no post-synthesis treatment is applied; yet, QY as high as 30.2% was observed. The very high QY could be attributed to a reduced particle size that causes more defects on the surface carbon, leading to more energy states to trap excitons during excitations (Pan, D., et al., Adv. Mater. 2010, 22, 734; Lingam, K., et al., Adv. Funct. Mater. 2013, 23:5062). It may also be due to addition of a non-carbon dopant, which is found recently as a cause of increased QY of C-dots (Sun, Y., et al., J. Phys. Chem. C 2008, 112, 18295; Tian, L., et al., Chem. Mater. 2009, 21:2803; Dong, Y., et al., Angew. Chem. Int. Ed. 2013, 52:7800).

Disclosed herein are MSN-templated synthetic methods to prepare Gd@C-dots. With the disclosed methods, different sizes of Gd@C-dots can be prepared in one-step, avoiding time-consuming purification that is required in conventional methods. In certain examples, 3.0 nm Gd@C-dots showed zero Gd leakage in physiological conditions, low toxicity, strong luminescence, high r₁ relaxivity, and efficient renal clearance, suggesting their great potential as a novel type of T₁ contrast agent. The disclosed methods can be extended to prepare other types of metal doped C-dots with good size and property control.

Specifically, disclosed herein is a method of forming nanoparticles of gadolinium encapsulated in an amorphous carbon shell, comprising calcining a mixture comprising a mesoporous silica nanoparticle, a gadolinium-containing compound, and a chelator, thereby forming the nanoparticles of gadolinium within the mesoporous silica nanoparticle; and removing the mesoporous silica nanoparticle from the nanoparticles of gadolinium. The chelators can form complexes with gadolinium and other metals. By calcining the metal-chelate complexes, a nanoparticle where the metal is encapsulated in an amorphous carbon shell can be produced. Calcining can be performed at from about 150° C. to about 300° C. in a muffle furnace or similar furnace. In specific examples, calcining can be performed at about 200° C.

Removing the mesoporous silica nanoparticle can be accomplished by dissolving it in a base and isolating the nanoparticles of gadolinium. Bases such as sodium hydroxide, potassium hydroxide, lithium hydroxide, calcium hydroxide, sodium carbonate, potassium carbonate, ammonium hydroxide, and the like can be used.

The mesoporous silica nanoparticle can be prepared by contacting a tetraalkyl orthosilicate and organofunctional silane in the presence of a tetraalkylammonium halide. Examples of suitable tetraalkyl orthosilicates are tetraethylorthosilicate, tetramethylorthosilicate, tetrapropylorthosilicate, and the like. Examples of organofunctional silanes aminosilanes include [3-(2-Aminoethylamino)propyl]trimethoxysilane, and the like. Examples of tetraalkylammonium halide include cetyltrimethylammonium bromide, cetyltrimethylammonium chloride, benzalkonium bromide, benzalkonium chloride, dodecyldimethyl ammonium bromide, dodecyldimethylammonium chloride, and other quaternary ammonium halides.

The mesoporous silica nanoparticle can have an average diameter of from about 100 nm to about 200 nm. For example, the mesoporous silica nanoparticles can have an average diameter of from about 110 nm to about 200 nm, from about 120 nm to about 200 nm, from about 130 nm to about 200 nm, from about 140 nm to about 200 nm, from about 150 nm to about 200 nm, from about 160 nm to about 200 nm, from about 100 to about 180 nm, from about 110 to about 160 nm, from about 120 nm to about 160 nm, or from about 130 nm to about 150 nm. In a specific example, the mesoporous silica nanoparticle can have an average diameter of about 150 nm.

The mesoporous silica nanoparticles can have an average pore size of from about 1 nm to about 20 nm. For example, mesoporous silica nanoparticles can have an average pore size of from about 1 to about 20 nm in diameter. For example, the disclosed mesoporous silica nanoparticles can have an average pore size of from about 2 nm to about 20, from about 4 nm to about 20 nm, from about 6 nm to about 20 nm, from 8 to about 20 nm, from about 10 nm to about 20 nm, from about 3 nm to about 20 nm, from about 3 nm to about 12 nm, from about 3 nm to about 10 nm, from about 3 nm to about 8 nm, from about 3 nm to about 6 nm, from about 4 nm to about 12 nm, or from about 4 nm to about 10 nm. In still other examples, the mesoporous silica nanoparticles can have an average pore size of 1, 2, 4, 6, 8, 9, 10, 11, 12, 13, 14, 15, 16, 17, 18, 19, or 20 nm, where any of the stated values can form an upper or lower endpoint of a range. In specific examples, the mesoporous silica nanoparticles can have an average pore size of about 3, about 7, or about 11 nm.

The nanoparticles of gadolinium have an average diameter about the same size as the pores of the mesoporous silica nanoparticles. For example, the nanoparticles of gadolinium can have an average diameter of from about 1 nm to about 20 nm. In other examples, the nanoparticles of gadolinium can have an average diameter of from about 2 nm to about 20, from about 4 nm to about 20 nm, from about 6 nm to about 20 nm, from 8 to about 20 nm, from about 10 nm to about 20 nm, from about 3 nm to about 20 nm, from about 3 nm to about 12 nm, from about 3 nm to about 10 nm, from about 3 nm to about 8 nm, from about 3 nm to about 6 nm, from about 4 nm to about 12 nm, or from about 4 nm to about 10 nm. In still other examples, the disclosed nanoparticles of gadolinium can have an average diameter of 1, 2, 4, 6, 8, 9, 10, 11, 12, 13, 14, 15, 16, 17, 18, 19, or 20 nm, where any of the stated values can form an upper or lower endpoint of a range. In specific examples, the nanoparticles of gadolinium can have an average diameter of about 3, about 7, or about 11 nm.

The chelator can be 1,4,7-triazacyclononane-1,4,7-triacetic acid (NOTA), 1,4,7,10-tetraazacyclodode-cane-1, 4, 7,10-tetraacetic acid (DOTA), 1,4,8,11-tetraazacyclododenane-1,4,8,11-tetraacetic acid (TETA), 2,2′-(1,4,8,11-tetraazabicyclo[6.6.2]hexadecane-4,11-diyl)diacetic acid (CB-TE2A), 3,6,9,15-Tetraazabicyclo[9.3.1]pentadeca-1(15),11,13-triene-3,6,9-triacetic acid (PCTA), pendetide (GYK-DTPA), cyclohexyldiethylenetriaminepentaacetic acid (CHX-DTPA), 2-(4,7-biscarboxymethyl[1,4,7]triazacyclonona-1-yl-ethyl)carbonyl-methylamino]acetic acid (NETA), diethylene triamine pentaacetic acid (DTPA), desferrioxamine, nitrilotriacetate (NTA), DO3A, ethylenediammine, acetylacetonate, phenanthroline, oxalate, citric acid, bipyridine, cyanide, nitrite, acetonitrile, ethylenediamine tetraacetic acid (EDTA), ethylene glycol tetraacetic acid (EGTA), poly-1-lysine, polyethylenimine, or polyvinylpyrrolidone (PVP), or any salt, derivative, functionalized analog, or mixture of these. In a specific example, the chelator can be diethylenetriaminepentacetate.

The methods can also involve conjugating the gadolinium nanoparticles with a targeting agent, a dye molecule, a metal chelate, or a drug molecule. For example, one can use N-(3-(dimethylamino)propyl)-N′-ethylcarbodiimide hydrochloride (EDC) and N-hydroxysuccininmide (NHS) chemistry to conjugate peptides, like cyclic RGD peptides, onto the gadolinium nanoparticles. A wide variety of natural and synthetic molecules recognized by target cells can be used as the targeting moiety. Suitable targeting moieties include, but are not limited to, a receptor, ligand, polynucleotide, peptide, polynucleotide binding agent, antigen, antibody, or combinations thereof. In one example, the targeting moiety is a peptide which has a length of from about 6 amino acids to about 25 amino acids.

### Compositions

Disclosed herein are nanoparticles where a metal is encapsulated in an amorphous carbon shell. The metal can be pure metal, metal oxide, metal complexes, or mixtures of these. These metal encapsulated carbon dots (herein referred to as M@C-dots) can have a wide variety of uses. In some specific examples, the metal is gadolinium; thus disclosed herein are Gd encapsulated carbon dots (hereafter referred to as Gd@C-dots). Also, reference to M@C-dots herein is meant to specifically include reference to Gd@C-dots.

Unlike most other nanocarriers/nanocapsules, carbon has low-toxicity and is highly biologically inert. Thus, the disclosed nanoparticle M@C-dots can remain intact even in harsh biological environments, therefore precluding the risk of metal release to the surroundings (Cao et al., Theranostics 2012, 2(3):295-301). With specific reference to gadolinium, stemming from the inert carbon coating, the disclosed nanoparticles are immune to the issue of Gd leaking that is often observed with complex-based Gd agents. Leakage of other metals from the disclosed nanoparticles is also expected.

The disclosed nanoparticles can have an average size of from about 1 to about 20 nm in diameter. For example, the disclosed nanoparticles can have an average size of from about 2 nm to about 20, from about 4 nm to about 20 nm, from about 6 nm to about 20 nm, from 8 to about 20 nm, from about 10 nm to about 20 nm, from about 3 nm to about 20 nm, from about 3 nm to about 12 nm, from about 3 nm to about 10 nm, from about 3 nm to about 8 nm, from about 3 nm to about 6 nm, from about 4 nm to about 12 nm, or from about 4 nm to about 10 nm. In still other examples, the disclosed nanoparticles can have an average size of 1, 2, 4, 6, 8, 9, 10, 11, 12, 13, 14, 15, 16, 17, 18, 19, or 20 nm nm, where any of the stated values can form an upper or lower endpoint of a range.

### Conjugates

The surface of the disclosed nanoparticles contains carboxyl groups that can be used to functionalize the surface of the nanoparticles. The carbonyl groups are electrophiles that can be used in nucleophilic substitution reactions or carbodiimide coupling reactions with any desirable functionalizing reagent. In certain examples, the functionalizing reagent can contain a targeting moiety that can be used to direct the functionalized nanoparticles to specific locations in the patient. Thus disclosed herein are M@C-dot nanoparticles functionalized with a targeting moiety. For example, RGD-peptides and cyclic RGD-peptides, when coupled to the disclosed M@C-dots, can direct the nanoparticles to target tumors. Similarly, EGFR targeting peptides, EGFR targeting therapeutics, VEGF targeting peptides, VEGF targeting therapeutics, and the like can be coupled/attached to the disclosed nanoparticles. Different types of antibodies, such as Herceptin, Avastin, and Erbitux, etc., can be coupled to the particle surface for facilitating particle targeting to tumors. Small molecule drugs such as doxorubicin, methotrexate or paclitaxel or their derivatives and can also be coupled to the surface of the nanoparticles, and in these cases the particles are used as drug carriers. Functionalizing the surface of the nanoparticles can also be used to assist the passage of the M@C-dots across certain cell membranes. For instance, the particle surface can be coated with a layer of positively charged polymer such as polyethylenimine and the resulting conjugates can be used as carriers for gene delivery (e.g., siRNA) due to assisting gene therapeutics passing through negatively charged cell membranes.

Suitable reagents for initiating a carbodiimide-mediate coupling to the carboxyl of the disclosed nanoparticles are commercially available. Specific examples of such reagents include, but are not limited to, water soluble carbodiimides such as 1-ethyl-3-(3-dimethylaminopropyl)-carbodiimide hydrochloride and 1-cyclohexyl-3-(2-morpholinoethyl)-carbodiimide-metho-p-toluene sulfonate, alcohol and water soluble N-ethyoxycarbonyl-2-ethoxy-1,2-dihydroquinoline, and organic soluble N,N′-dicyclohexylcarbodiimide.

### Formulations

While it can be possible for disclosed nanoparticles to be administered neat, it is also possible to present them as a pharmaceutical formulation. Accordingly, provided herein are pharmaceutical formulations which comprise one or more of the disclosed nanoparticles together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients can be used as suitable and as understood in the art; e.g., in Remington: The Science and Practice of Pharmacy, 21^{st} Ed., Gennaro, Ed., Lippencott Williams & Wilkins (2003). The compositions and formulations disclosed herein can be manufactured in any manner known in the art, e.g., by means of conventional mixing, dissolving, granulating, dragee-making, levigating, emulsifying, encapsulating, entrapping or compression processes.

A nanoparticle as disclosed herein can be incorporated into a variety of formulations for therapeutic administration, including solid, semi-solid, or liquid forms. The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route can depend upon for example the condition and disorder of the recipient. The formulations can conveniently be presented in unit dosage form and can be prepared by any of the methods well known in the art of pharmacy. Typically, these methods include the step of bringing into association a compound or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

The disclosed nanoparticles can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. The formulations can be presented in unit-dose or multi-dose containers, for example sealed ampoules and vials, and can be stored in powder form or in a freeze-dried (lyophilized) condition requiring only the addition of the sterile liquid carrier, for example, saline or sterile pyrogen-free water, immediately prior to use. Extemporaneous injection solutions and suspensions can be prepared from sterile powders, granules and tablets of the kind previously described.

Formulations for parenteral administration include aqueous and non-aqueous (oily) sterile injection solutions of the active compounds which can contain antioxidants, buffers, bacteriostats and solutes which render the formulation isotonic with the blood of the intended recipient; and aqueous and non-aqueous sterile suspensions which can include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Nanoparticle Synthesis and Characterizations

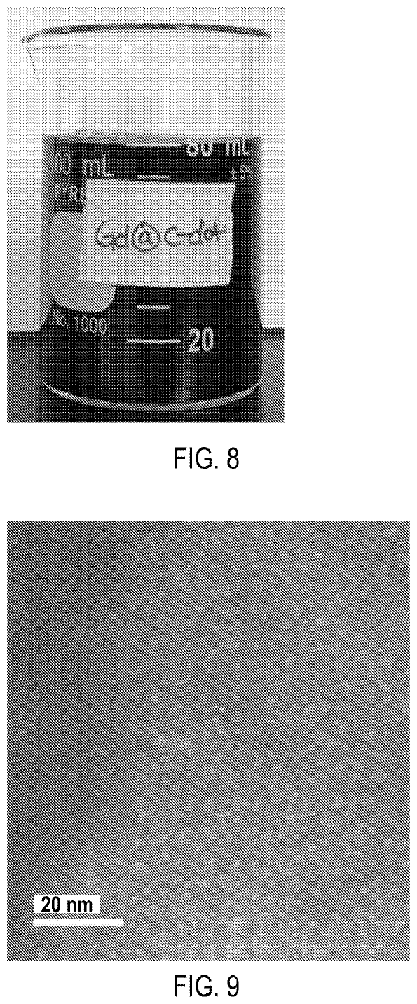

Gd@Cdots were synthesized through a hydrothermal reaction. Briefly, p-phenylenediamine (pPD) and Gd (NO₃)₃ were dissolved in EtOH, and the solution was transferred into an autoclave. The reaction took place at 180° C. for 12 h. After reaction, we collected nanoparticles by centrifugation and subjected them to dialysis to remove unreacted precursors and surface-bound metals. The purified products were re-suspended in water, forming a transparent, wine-colored solution (FIG. 8).

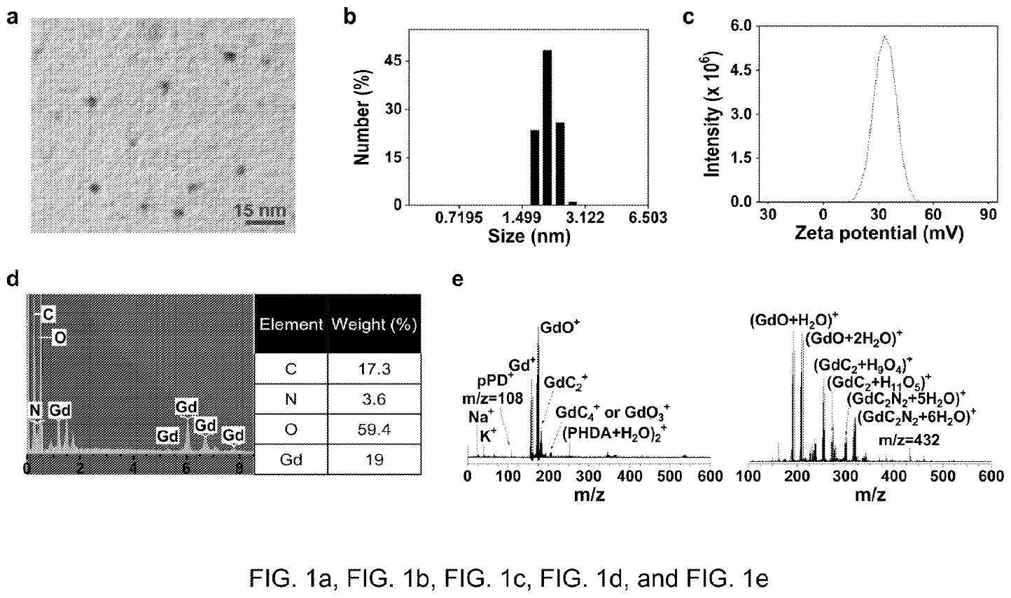

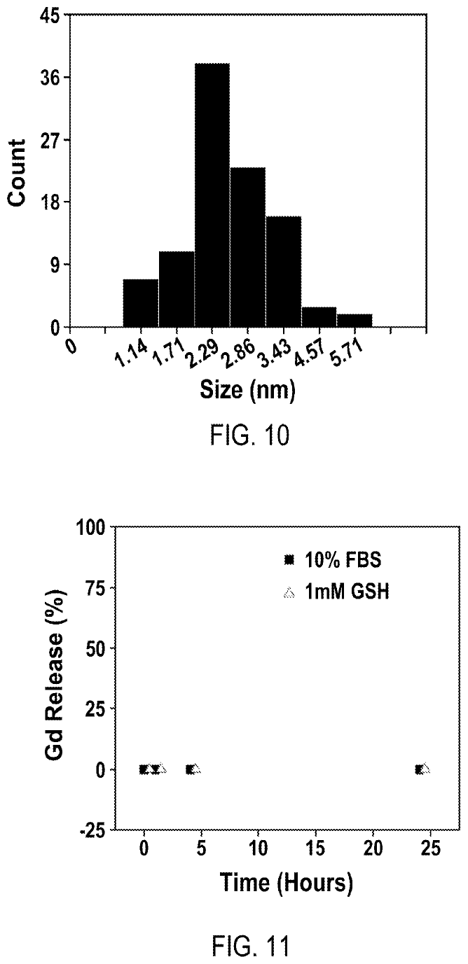

The size and morphology of the nanoparticles was analyzed by transmission electron microscopy (TEM, FIG. 1a) and scanning transmission electron microscopy (STEM, FIG. 9). The average nanoparticle size was 2.60 nm, with a relatively narrow size distribution (FIG. 10). Dynamic light scattering (DLS) confirmed that the nanoparticles were 2-3 nm in diameter with a polydispersity index (PDI) of 0.364 (FIG. 1b). Zeta potential analysis showed that the nanoparticle surface was positively charged (+33.3 mV, FIG. 1c), which is due to surface amine groups inherited from pPD.

The composition of Gd@Cdots was investigated by energy dispersive spectroscopy (EDS, FIG. 1d). A high Gd content was observed, with a Gd/C molar ratio of 0.09:1 (FIG. 1d). Moreover, a significant amount of nitrogen was found in the particles, which was mainly attributed to the surface amine groups. We further probed the nanoparticle composition by mass spectroscopy (MS) using a time-of-flight (TOF) detector. FIG. 1e shows the MS spectra of Gd@Cdots after laser desorption and electrospray ionization. Oxidized Gd clusters were detected and assigned to GdC₂⁺ clusters or their water/N₂ solvated clusters (FIG. 1e). It is reasoned that Gd is tightly bound to the carbon matrix that is fragmented during laser desorption or ionization, forming clusters. Fragmentation and successive loss of C₂ units is common among carbon species such as fullerenes.

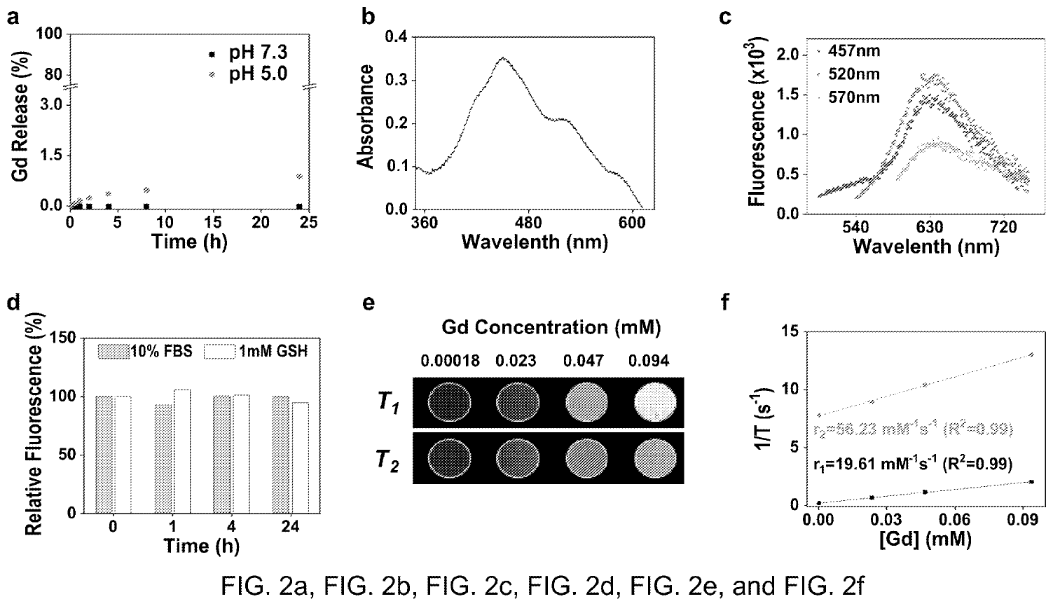

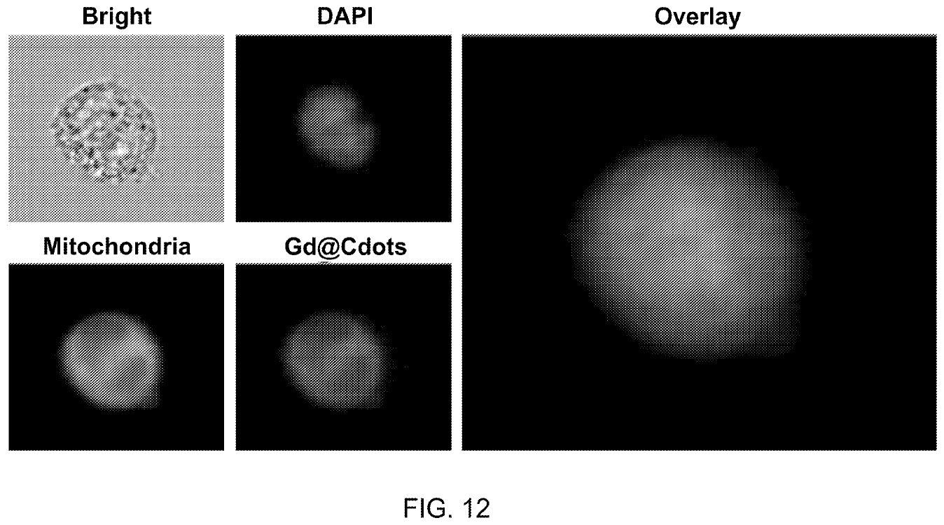

The stability of Gd@Cdots was assessed by analyzing Gd³⁺ released from the nanoparticles in different solutions. In both neutral and acidic buffer solutions (pH=7.4 and 5.0, respectively), less than 1% Gd was released over 24 h incubation (FIG. 2a). Minimal Gd³⁺ release was also observed when nanoparticles were incubated in the serum or glutathione solutions (GSH, FIG. 11). The low Gd leakage was attributed to the inert carbon coating that prevents metal escape.

Gd@Cdots show three distinct absorbance peaks at 457, 520, and 570 nm (FIG. 2b). This is different from Gd@Cdots or Cdots made from calcination, which often show broad absorbance across the visible spectrum region. Gd@Cdots also display intense fluorescence at ˜ 635 nm (FIG. 2c&d), which potentially benefits imaging. As observed in previous studies, Gd@Cdots afford strong magnetic properties. This was demonstrated in a phantom study, where Gd@Cdots of different concentrations were dispersed in 1% agarose gel and scanned on a 7T magnet (FIG. 2e). Gd@Cdots showed concentration-dependent signal increase on T₁ images. Based on region of interest (ROI) analysis and linear regression fitting, it was determined that the r₁ relaxivity of Gd@Cdots was 19.6 mM⁻¹s⁻¹, and the r₂/r₁ ratio was 2.87 (FIG. 2f).

### Example 2: Radical Production Under Radiation

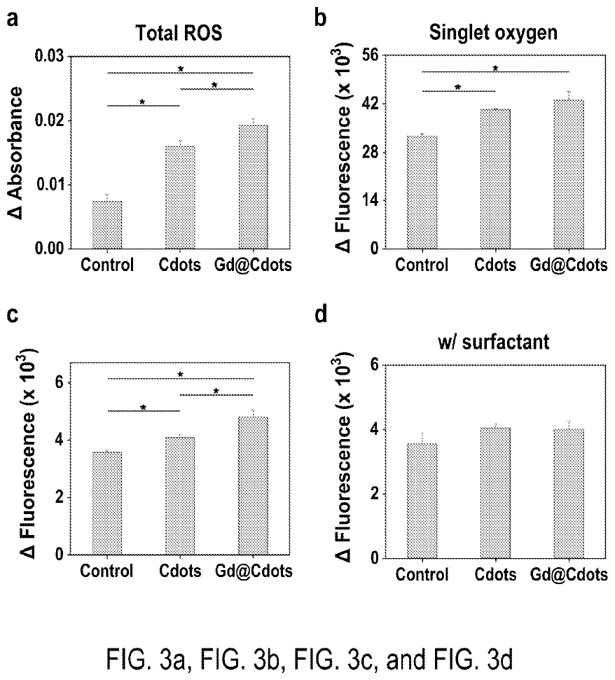

We then examined whether Gd@Cdots can enhance radical production under beam radiation. This was tested in a Tris buffer solution (pH 7.4) using methylene blue, singlet oxygen sensor green (SOSG), and terephthalic acid (TA) as radical probes. While methylene blue can be bleached by a wide range of radicals, SOSG and TA are selective flurogenic probes for singlet oxygen (¹O₂) and hydroxyl radicals (·OH), respectively. Relative to the control, Gd@Cdots (30 μg/mL) significantly increased the degree of methylene blue bleaching under irradiation (5 Gy, FIG. 3a). Meanwhile, fluorescence signals of SOSG and TA were both increased in solutions containing Gd@Cdots (FIG. 3b&c), suggesting that the particles enhance reactive oxygen species (ROS) production under radiation.

For comparison, carbon dots (Cdots) of the same size were prepared and tested. Interestingly, in Cdots solutions, ¹O₂ and ·OH were also elevated under radiation, although the amplitude of increase was less prominent than in Gd@Cdots solutions (FIG. 3b). This observation suggests that in addition to gadolinium's photoelectric effects, the carbon shell may have played a role in boosting radical generation. We reason that this enhancement is attributed to the surface catalytic effects that promote ionization of molecules such as water. Specifically, with a large surface area and multiple surface amine groups, Gd@Cdots/Cdots may form hydrogen bonds with surrounding water molecules, thus weakening the intramolecular H—OH bond and facilitating water radiolysis. To investigate, we repeated the TA study but adding a surfactant, Triton X-100, into the Gd@Cdots solution to break the hydrogen bond between the particles and water. We found a significantly decreased TA fluorescence, supporting the postulation that surface catalytic effects contribute to radical production (FIG. 3d).

### Example 3: Cellular Uptake and Cytotoxicity

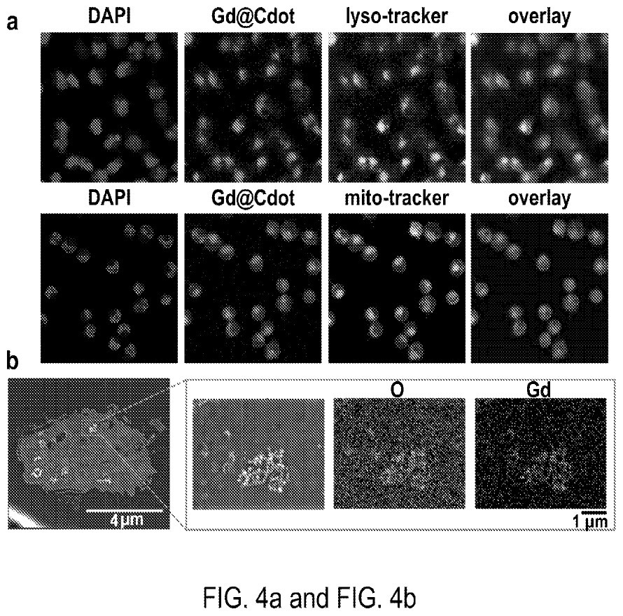

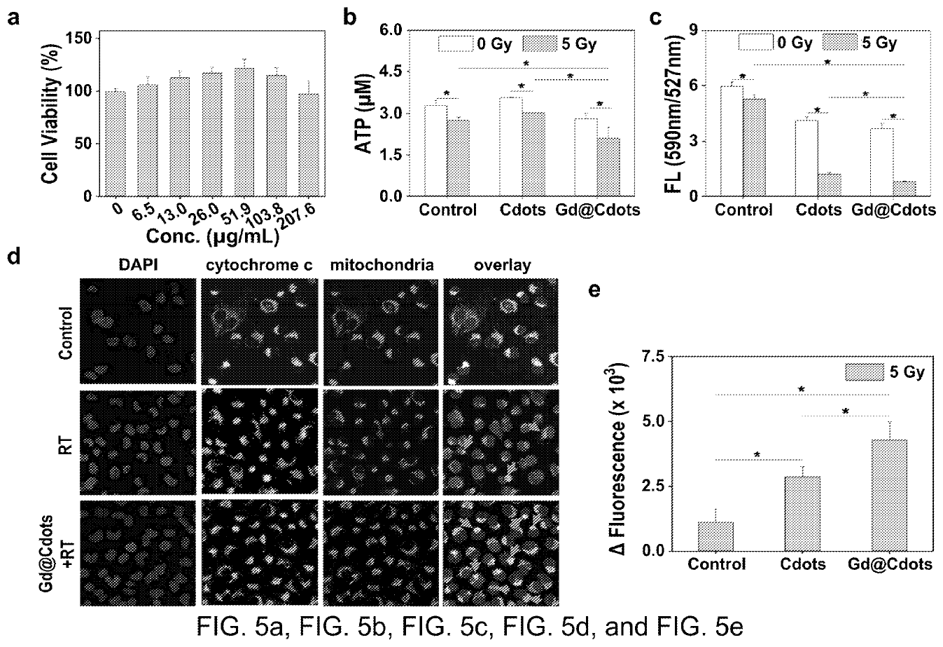

We next examined Gd@Cdots uptake by cancer cells. We tested this in H1299 cells, which are a human NSCLC cell line. Gd@Cdots can be readily traced under a microscope due to intrinsic fluorescence. We observed a good signal overlap between LysoTracker and Gd@Cdots, suggesting that Gd@Cdots enter cells through endocytosis (FIG. 4a). This was supported by STEM analysis of sectioned cell samples, which found many nanoparticles in the endosomes/lysosomes (FIG. 4b). Meanwhile, MitoTracker staining revealed that a significant amount of Gd@Cdots were accumulated in the mitochondria (FIGS. 4a and 12). This is attributed to Gd@Cdots' compact size and their positive surface charge, both factors benefiting mitochondrion translocation and retention. We then assessed the cytotoxicity of Gd@Cdots by MTT assay. No significant viability drop was observed even at high Gd concentrations (e.g. 1.32 mM, FIG. 5a). This low toxicity is consistent with our previous observations, and is attributable to minimal Gd leakage.

### Example 4: Cytotoxicity and Clonogenicity Studies

We further assessed the impact of Gd@Cdots on radiation-induced cell killing. ATP bioluminescence assay found that Gd@Cdots plus radiation (Gd@Cdots+RT, 5 Gy) caused a significant drop of cell viability by 36.1%, compared to 16.3% for RT alone (FIG. 5b). Considering that Gd@Cdots accumulate in mitochondria, we postulate that the particles may promote damage to the organelle. Indeed, JC-1 staining found that Gd@Cdots+RT led to a significant decrease of mitochondrial membrane potential (ΔΨm) relative to RT alone (manifested in a decrease of red-to-green fluorescence ratio, FIG. 5c). The mitochondrial depolarization in turn caused cytochrome c translocation to the cytosol (FIG. 5d) and activation of the intrinsic apoptosis pathway, which was evidenced by increased caspase 3 activity (FIG. 5e).

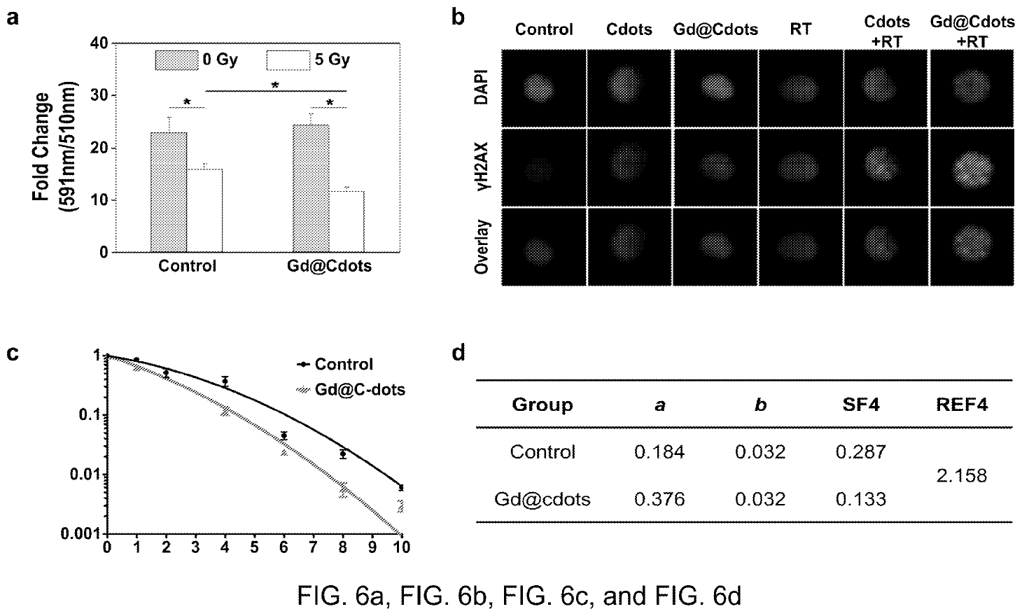

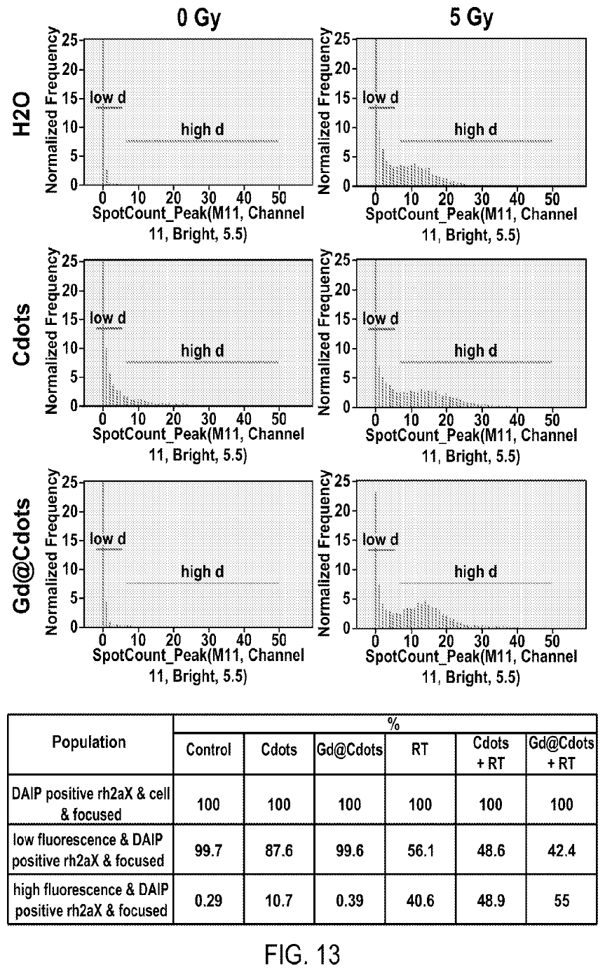

Mitochondrion damage would also exacerbate oxidative stress in cells and in turn cause extensive damage to other cellular components. For instance, BODIPY fluorogenic assay showed that cell lipid peroxidation level was increased by 49.4% in the presence of Gd@Cdots (FIG. 6a). γH2AX staining identified an increased foci number, indicating enhanced DNA damage with Gd@Cdots+RT (FIGS. 6b and 13). It is worth mentioning that some Gd@Cdots were found inside cell nuclei (FIG. 4a), which may have contributed to the double-strand breaks.

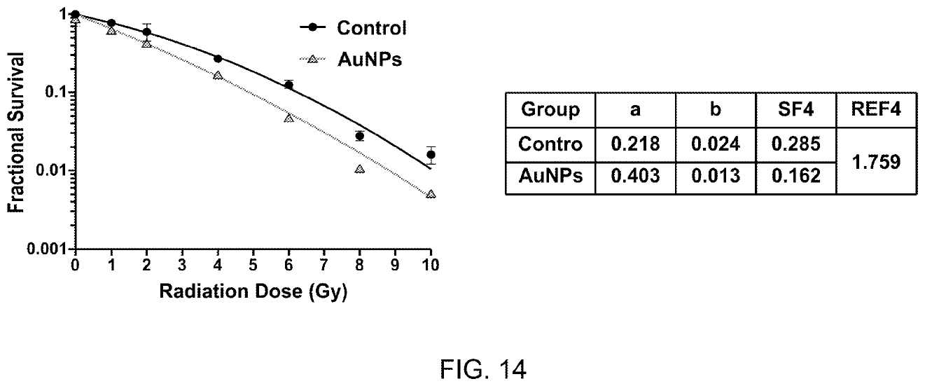

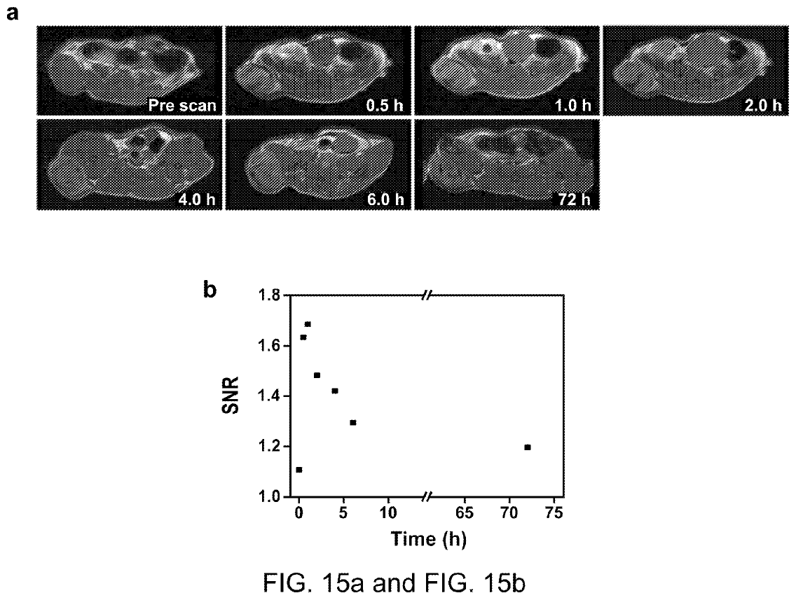

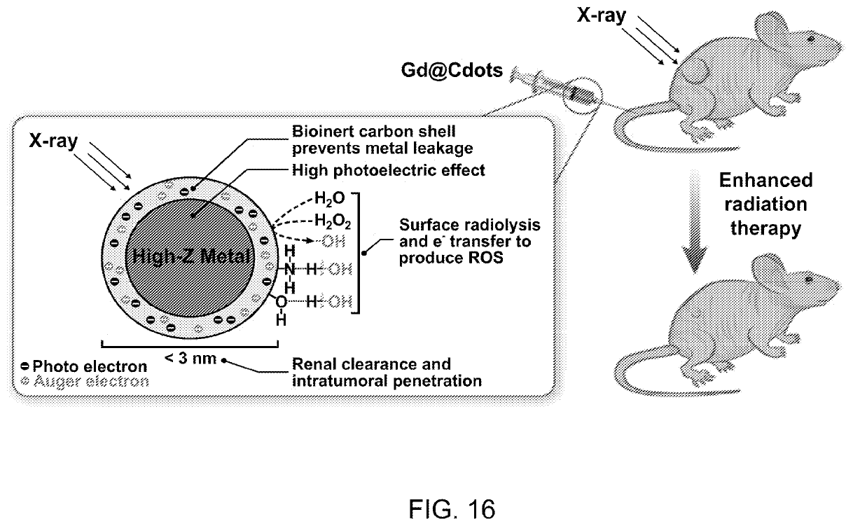

Last but not least, we assessed the dose-modifying effects of Gd@Cdots by clonogenic assay. Briefly, H1299 cells were incubated with Gd@Cdots (10 μg·Gd/mL) or PBS and then subjected to radiation at elevated doses (0-10 Gy). The treated cells were then seeded onto a petri-dish, and after 14 days, colonies with more than 50 cells were counted. The results were fitted into the linear-quadratic equation (FIGS. 6c&d). The presence of Gd@Cdots significantly enhanced the efficacy of RT at all tested doses. The survival fraction at 4 Gy, or SF4, was 0.133 for the Gd@Cdots+RT group, compared to 0.287 in the RT-only control. This represents a radiation enhanced factor at 4 Gy, or REF, of 2.158. For comparison, gold nanoparticles at the same metal concentration were also tested. Although Au is a much heavier atom (Z=79, compared to 64 of Gd), gold nanoparticles showed a lower REF of 1.759 (FIG. 14). The superior radiosensitizing effects are attributed to Gd@Cdots' surface effects and possibly their accumulation in critical organelles such as the mitochondria and nuclei.

### Example 5: In Vivo Therapy Studies

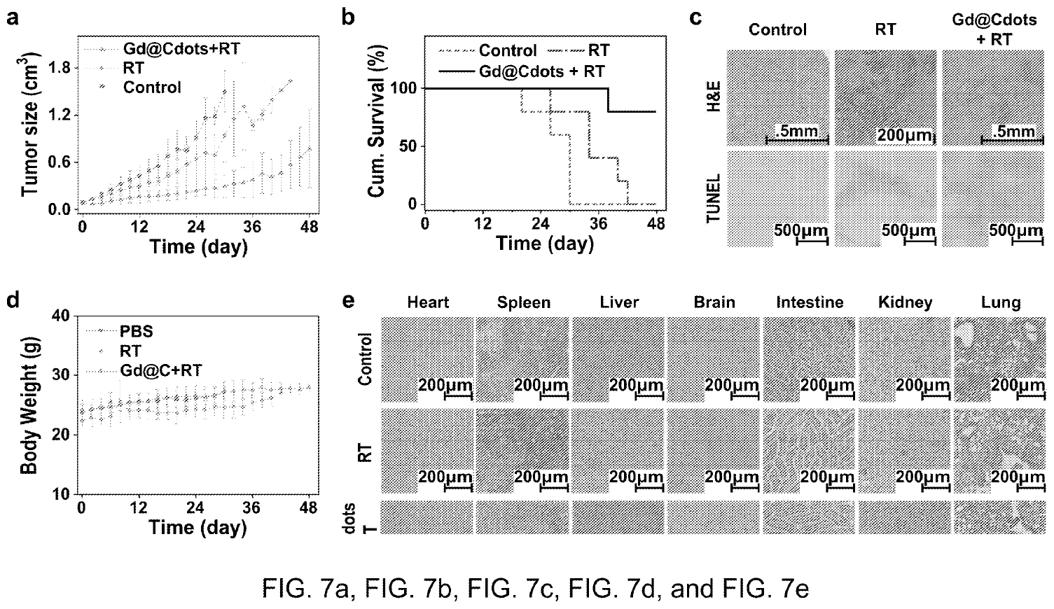

The benefits of Gd@Cdots for RT were further evaluated in vivo in a H1299 xenograft model. The animals were randomly divided to receive treatments by Gd@Cdots plus radiation (Gd@Cdots+RT), radiation only (RT), or PBS only (n=5). For the Gd@Cdots+RT group, Gd@Cdots at 0.1 mmol Gd/kg were i.v. injected, and X-ray (6 Gy) in a single beam was applied to tumors at 4 h, with the rest of the body lead-shielded. For the RT only group, the same radiation dose was applied. Relative to the PBS control, Gd@Cdots+RT led to a tumor inhibition rate (TIR) of 80.3% on Day 30 (FIG. 7a). By the end of the study (Day 48), 80% of the animals in the Gd@Cdots+RT group remained alive (FIG. 7b). As a comparison, RT showed a mediocre TIR of 37.2% on Day 30 (FIG. 7a), with all the animals reaching a humane endpoint by Day 42 (FIG. 7b). Post-mortem H&E and TUNEL staining found a reduced level of cancer cell density and an increased level of apoptosis in tumors taken from the Gd@Cdots+RT group (FIG. 7c), confirming the radiosensitizing effects of Gd@Cdots.

Meanwhile, no body weight drop was observed throughout the experiment for the Gd@Cdots+RT group (FIG. 7d). After euthanizing the animals, major organs, including the heart, spleen, liver, brain, intestine, kidneys, and lung, were harvested and analyzed by histology. H&E staining found no sign of toxicity in all tested organs (FIG. 7e). For better assessment of toxicity, in a separate study, Gd@Cdots (0.1 mmol/kg) were injected into healthy mice, and blood samples were collected on Day 14 for analysis. Complete blood count (CBC) and biochemistry analyses found no significant difference between the Gd@Cdots and PBS groups in all tested indices (Table 1). BUN and ALT levels were also in the normal ranges (Table 1), confirming low toxicity.

TABLE 1
Complete blood count (CBC) results as well as liver & kidney function tests

| | | Reference | | Experiment | | | |
|---|---|---|---|---|---|---|---|
| | | (95% interval) | | Control | | Gd@Cdot | |
| | Unit | Low | High | Average | Std | Average | Std |
| RBC | ×10\hat{ }6/ul | 8.16 | 11.69 | 11.75 | 0.21 | 11.10 | 0.26 |
| HGB | g/dl | 12.40 | 18.90 | 18.50 | 0.14 | 17.30 | 0.61 |
| HCT | % | 43.50 | 67.00 | 54.60 | 1.84 | 52.67 | 2.06 |
| MCV | fl | 50.80 | 64.10 | 46.60 | 0.85 | 47.57 | 0.67 |
| MCH | pg | 13.00 | 17.60 | 15.75 | 0.07 | 15.63 | 0.47 |
| MCHC | g/dl | 23.90 | 33.10 | 33.85 | 0.78 | 32.40 | 1.27 |
| RDW | % | 16.90 | 23.50 | 13.75 | 0.35 | 14.10 | 0.17 |
| Platelets | ×10\hat{ }3/ul | 476.00 | 1661.0 | 229.50 | 94.05 | 538.67 | 250.23 |
| MPV | fl | 4.60 | 5.80 | 5.55 | 0.49 | 6.17 | 0.15 |
| WBC | ×10\hat{ }3/ul | 5.69 | 14.84 | 6.00 | 1.41 | 7.03 | 0.45 |
| Neutrophils | ×10\hat{ }3/ul | 0.74 | 3.01 | 1.27 | 0.11 | 1.38 | 0.41 |
| Lymphocytes | ×10\hat{ }3/ul | 3.60 | 11.56 | 4.03 | 1.24 | 5.18 | 0.56 |
| Monocytes | ×10\hat{ }3/ul | 3.75 | 14.33 | 0.10 | 0.06 | 0.23 | 0.07 |
| Eosinophils | ×10\hat{ }3/ul | 0.01 | 0.35 | 0.34 | 0.41 | 0.12 | 0.05 |
| Basophils | ×10\hat{ }3/ul | 0.00 | 0.16 | 0.27 | 0.18 | 0.12 | 0.04 |
| BUN | mg/dL | 7.00 | 31.00 | 24.39 | 4.19 | 21.17 | 3.71 |
| ALT | unit/L | 40.00 | 170.00 | 58.29 | 3.63 | 54.10 | 1.50 |

### Materials and Methods Used in Examples

Materials

P-phenylenediamine (pPD) (Sigma Aldrich, Cat #78429), gadolinium nitrate hexahydrate (Gd(NO₃)₃·6H₂O, Sigma Aldrich, Cat #211591), ethanol (KOPTEC, Cat #19J14D), dialysis membrane (Spectrum, MWCO=100-500), Milli-Q H₂O, 3-(4,5-dimethylthiazolyl-2)-2,5-diphenyltetrazolium bromide (MTT) (Sigma Aldrich, Cat #M2128).

Gd@Cdots Synthesis

Gd@Cdots were synthesized by a hydrothermal method following our previous publication.¹⁸ Briefly, 0.16 g of pPD and 0.6 g of Gd(NO₃)₃ were dissolved in 60 mL EtOH, and the solution was transferred into a 100 ml poly(tetrafluoroethylene)-lined stainless steel autoclave. The reaction was heated at 180° C. for 12 h and cooled down to room temperature. The resulting dark red suspension was purified using dialysis membrane (MWCO 500) against to Milli-Q water for 17 h to remove bi-carbon products and extra Gd³⁺ free ions. The final product was freeze dried for further experiment and long-term storage.

Physical Characterizations

Transmission electron microscopy (TEM) was carried out on a FEI TECNAI 20 transmission electron microscope at 200 kV. The absorbance and fluorescence spectra were obtained on a BioTek Synergy MX multi-mode microplate reader. Scanning transmission electron microscopy (STEM) image was obtained using FEI G2 TECNAI F30 at 300 kV. The zeta potential and size distribution measurements were carried out on a Malvern Zetasizer Nano ZS system (Zeta potential+33.3 mV, DLS 2.01 nm). Energy-dispersive X-ray spectroscopy (EDS) and element mapping were performed on a FEI Inspect F FEG-SEM equipped with EDZX EDS system to confirm Gd contents in the Cdots. Inductively coupled plasma mass spectrometry (ICP-MS) was used to analyze the Gd concentration in the sample for further study.

Mass Spectrum Analysis

We collected the mass spectra using both laser desorption ionization (LDI) and electrospray ionization (ESI) mass spectroscopy. ESI mass spectroscopy was performed on a Waters LCT Premier mass spectrometer. MassLynx was used as the software to collect spectra. Each ESI mass spectrum shown in this work was an average of 55 mass spectra collected in 1 minute with 0.1 second between every two one-second scans. Samples were diluted about 10 times before injection. For LDI measurements, we used the linear mode of the Comstock RTOF-210 mass spectrometer with a pulsed Nd:YAG laser at 355 nm (New Wave Research Polaris II). The laser power was less than 400 μJ/pulse. All the LDI mass spectra exhibited here were averaged from 200˜500 scans. Sample solutions were applied to a solid copper tip, dried in air to form a thin film, and then inserted into the ion source.

Physical Stability of Gd@Cdots

The Gd@Cdots were incubated in PBS at different pH (pH=5.0 and 7.2) to test the stability of the particles and the release of Gd³⁺. The samples were kept in an incubating shaker at 37° C. At each time point (0, 0.5, 1, 2, 4, 8 and 24 h), sample solutions were collected and centrifugated on micro-filter units (MWCO: 3k; Amicon® Cat #UFC800308). Solutions passing through the membrane was analyzed by ICP-MS to evaluate free Gd³⁺.

Optical Properties of Gd@Cdots

Gd@Cdots were dispersed in Milli-Q H₂O (100 μg/mL) and transferred to a quartz cuvette. Absorbance between 200-800 nm was scanned on a Varian Cary 300 bio UV-visible spectrometer. For emission spectrum, Gd@Cdots were dispersed in Milli-Q H₂O (100 μg/mL) and placed in a black 96-well plate (Corning Costar, Cat #3614). The fluorescence spectra were acquired on a microplate reader (Synergy Mx, BioTeK) with excitation at 457, 520, and 570 nm.

MRI Phantom Studies

MRI phantom samples were prepared by dispersing Gd@Cdots (0-0.1 mM) in 1% (w/w %) agarose gel. T₁ and T₂ images were acquired on a Varian Magnex 7 Tesla scanner. For T₁-weighted images, a T₁ inversion recovery fast spin echo (FSE) sequence was used using the following parameters: TR=5000 ms, ESP=7.69, Segment/ETL=32/8, Effective TE=30.75 ms, inversion times (TI)=10.00-1500.0 ms with array size of 8, 256×256 matrices. For T₂-weighted images, a FSE sequence was used with following parameters: TR=2000 ms, TE=8.00 ms, NE=12, 256×256 matrices.

Reactive Oxygen Species Analyses

Overall ROS generation was evaluated using methylene blue assay. Briefly, a series of Gd@Cdots (20, 60, and 120 μg/mL, based on Gd content, the same below) and 60 μg/mL of methylene blue were prepared in Tris Buffer (pH=7.4). A 100 μL solution of Gd@Cdots and a 100 μL solution of methylene blue were added to a 96-well plate (Corning Costar, Cat #3599), making the final Gd concentrations being 10, 30, and 60 μg/mL. The initial absorbance was measured on a microplate reader (Synergy Mx, BioTeK). The Gd@Cdots methylene blue solution was irradiated with 5 Gy X-ray. The absorbance after irradiation was measured and compared to the initial absorbance. The difference was computed and used to evaluate overall reactive oxygen species generation.

Singlet oxygen (¹O₂) and hydroxyl radical (·OH) were measured using Singlet Oxygen Sensor Green (SOSG, Invitrogen™ Cat #S36002) and Terephthalic Acid (TA, Signa Aldrich, (Cat #185362), respectably. Briefly, a series of Gd@Cdots (20, 60 and 120 μg/mL), 2 μM of SOSG, and 16 mM of TA solution were prepared in Tris buffer solutions. A 100 μL solution of Gd@Cdots, and a 100 μL chemical sensor solution (SOSG or TA) were mixed and added to a 96-well plate (Corning Costar, Cat #3614); the final Gd concentrations were 10, 30 and 60 μg/mL. The initial fluorescence was measured on a microplate reader (Synergy Mx, BioTeK). The Gd@Cdots solutions received 5 Gy irradiation and the fluorescence was measured again. The variation in fluorescence intensity was computed to evaluate singlet oxygen and hydroxyl radical production. To test whether the surface chemical functional groups of Gd@Cdots facilitate radical production, nanoparticles were incubated in solutions containing 0.1% Triton X-100 before mixing with TA and receiving irradiation.

Cell Uptake Studies

H1299 cells, which originated from human non-small lung cancer tumors, were cultured by following a protocol provided by ATCC. Gd@Cdots co-localization in the lysosome and mitochondria were tested using LysoTracker™ Green DND-26 (ThermoFisher, Cat #L7526) and MitoTracker™ Green FM (ThermoFisher, Cat #M7514), respectably. Briefly, 1×10⁶ of H1299 cells were seeded on 2-chamber glass slide (Nunc™ Lab-Tek™ II Chamber Slide™ System, ThermoFisher) and incubated with Gd@Cdots at 37° C. for 4 h. After the cells were washed with PBS for 3 times, 20 nM LysoTracker or MitoTracker was added to stain the lysosome or the mitochondria for 30 min, respectably. The cells were fixed with 4% formaldehyde, and cell nuclei were stained with DAPI. Fluorescence images were taken on a Zeiss LSM 710 Confocal Microscope with 40× magnification.

Scanning Transmission Electron Microscopy

To study the intra-cellular distribution of the particle, cells were also imaged by scanning transmission electron microscopy (STEM, FE-SEM FEI Teneo). Briefly, H1299 cells were incubated with (Gd@Cdots for 4 h. The cells were collected, fixed with glutaraldehyde, and sectioned into thin slices. Resulting samples were loaded onto a carbon grid. The distribution of Gd(@Cdots was evaluated on a FE-SEM Thermo Fisher Teneo system with EDS mapping.

Cytotoxicity

Cell viability was studied with H1299 cells using standard MTT and ATP bioluminescence assays. For MTT assays, H1299 cells (8000 cells per well) were seeded onto a 96-well plates (Corning Costar, Cat #3599). When cells were attached, Gd@CDots at a final concentration of 0-207.6 μg/mL were added into the wells and incubated with cells for 24 h. A 20 μL solution of 10 mg/mL 3-(4,5-dimethylthiazolyl-2)-2,5-diphenyltetrazolium bromide was added into each well. After 4 h, the solution was aspirated, and 100 μL of DMSO was added to each well. The absorbance at 570 nm was measured on a BioTek Synergy MX multi-mode microplate reader. For ATP assays, cells were incubated with Gd@Cdots (60 μg/mL), Cdots (60 μg/mL), or PBS for 4 h, followed by 5 Gy irradiation. After 24 h incubation, the supernatant was completely removed, and 55 μL cell culture medium and 55 μL ATP kit solution were added. Solution from each well was transferred to a new, opaque 96-well plate and luminescence signal was measured on a microplate reader (Synergy Mx, BioTeK). The result was compared to a standard curve established according to the manufacture's protocol.

Mitochondrial Membrane Potential (ATm)

ΔΨₘ change was assessed by JC-1 staining (Biotium, Cat #30001). The JC-1 working solution was prepared by adding 10 μL of the concentrated dye to 1 mL of FBS free RPMI medium. 200 μL of cell culture medium containing Gd@Cdots (30 μg/mL) or PBS was incubated with cells for 4 h. The cells were irradiated with 5 Gy and incubated for 24 h. The medium was removed and replaced with the JC-1 working solution. After 15 min incubation, the fluorescence signals of the stained cells were measured on a microplate reader (green: ex/em 510/527 nm; red: ex/em 585/590 nm), and the green-to-red fluorescence intensity ratio was computed.

Cytochrome c Release

Cytochrome c release was evaluated using ApoTrack™ Cytochrome c Apoptosis ICC Antibody Kit (Abcam, Cat #ab110417). Briefly, 1×10⁶ of H1299 cells were seeded onto 2-well chamber slide for attachment. The cells were then incubated with Gd@Cdots (30 μg/mL) or PBS for 4 h before receiving 5 Gy irradiation. After 24 h, antibody was added following the manufacture's protocol. Images were taken on a Zeiss LSM 710 Confocal Microscope and analyzed by ImageJ.

Caspase-3 Activity

For caspase-3 activity measurement, H1299 cells were incubated with Gd@Cdots (30 μg/mL) or PBS for 4 h, followed by 5 Gy irradiation. After 24 h incubation, cells were stained using FAM-FLICA® Caspase-3/7 kit (Immunochemistry, Cat #94) following the manufacturer's protocol. The caspase-3 activity was evaluated by measuring fluorescence signals (ex/em: 488/530 nm) on a microplate reader (Synergy Mx, BioTeK).

Lipid Peroxidation Assay

Image-iT Lipid Peroxidation Kit (Abcam, Cat #ab118970) was used to assess lipid peroxidation. Briefly, cells were pre-seeded onto a 96-well plate and incubated with Gd@Cdots (30 μg/mL), Cdots (30 μg/mL), or PBS at 37° C. for 24 h. After replenishing medium, cells received 5 Gy irradiation. After 24 h, cells were stained with Image-iT Lipid peroxidation sensor (30 μM) for 30 minutes at 37° C., and washed with PBS for three times. The yellow and green fluorescence intensities (ex/em: 581/591 nm and 488/510 nm, respectively) were recorded on a microplate reader (Synergy Mx, BioTeK), and the ratio between them was computed.

rH2AX Assay

The DNA damage was evaluated using anti-rH2AX (Alexa 647) antibody (Millipore Sigma, Cat #07-164-AF647). Briefly, H1299 cells were pre-seeded onto a 35-mm cell culture dish and incubated with Gd@Cdots (30 μg/mL), Cdots (30 μg/mL), or PBS. After 4 hour incubation, cells received 5 Gy irradiation and continued incubation for another 1 h at 37° C. The cells were then collected, fixed, and permeabilized, and stained with anti-rH2AX antibody according to the protocol from the manufacture. Cells with positive anti-rH2AX stain was analyzed using a Millipore Sigma ImageStream X Mark II Imaging Flow Cytometer.

Clonogenic Assay

Briefly, H1299 cells were pre-seeded onto a 35-mm cell culture dish (Corning, Cat #430165) and incubated with Gd@Cdots (10 μg/mL) or PBS for 12 h. After washing, cells were collected and seeded (100-10000 cells, depending on the radiation dose) onto a 100-mm plate (Falcon, Cat #353003), and irradiated (0-10 Gy). After 14 days, colonies were stained with crystal violet and counted. Data were fit into the linear-quadratic model: S(D)/S(0)=exp−(aD+bD²), where S is cell survival fraction, D is radiation dose, and a&b are fitting coefficients.

In Vivo Radiation Therapy

In vivo therapy studies were performed on H1299 subcutaneous tumor models established on 4-week-old female nude mice purchased from Charles River. All animal experiments were performed according to a protocol approved by the Institutional Animal Care and Use Committee (IACUC) of the University of Georgia. The tumor model was developed by subcutaneous injection of 2.5×10⁶ H1299 cells into the right flank of mice. When the tumor size reached 100 mm³, the mice were randomly divided into three groups (PBS, PBS+RT, and Gd@Cdots+RT). The radiation was delivered through an X-RAD 320 system. Gd@Cdots were intravenously injected (0.1 mmol/kg, 200 μL); after 4 hours, tumors received 6 Gy radiation, with the rest of the animal body lead-shielded. The tumor size was measured every 2 days with a caliper, and the tumor volume was calculated using the equation: tumor volume=(tumor length×tumor width²)/2). The mice were euthanized when a humane end point was reached. Tumors and major organs such as the brain, liver, heart, lung, intestine, kidney, and spleen were collected for hematoxylin and eosin (H&E) and TUNEL staining.

Complete Blood Counts and Biochemistry Analysis

In a separated experiment, three balb/c mice were intravenously injected with PBS or Gd@Cdots (0.1 mmol Gd/kg). Blood samples were collected using a cardiac puncture blood collection method. 250 μL of blood samples was subjected for complete blood counts. The remaining blood samples were used to evaluate liver and kidney function using Alanine Aminotransferase (ALT) ELISA kit (Abcam, Cat #ab105134) and Urea Nitrogen (BUN) detection kit (Arbor Assays, Cat #K024H1), respectively.

Statistical Analysis

Graphpad Prism 8 software (GraphPad Software, San Diego, CA) was used for statistical analysis. For reactive oxygen species studies and all the in vitro studies, data were expressed as mean±standard deviation. For in vivo study, each group had 5 animals (n=5). Statistical significance was evaluated by one-way ANOVA or two-way ANOVA with multiple comparison. The statistical significance was set at *p<0.05.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

A. Detappe, E. Thomas, M. W. Tibbitt, S. Kunjachan, O. Zavidij, N. Parnandi, E. Reznichenko, F. Lux, O. Tillement and R. Berbeco, Nano Lett., 2017, 17, 1733-1740.
A. Jemal, R. Siegel, J. Xu and E. Ward, CA Cancer J. Clin., 2010, 60, 277-300.
A. Rajaee, S. Wang, L. Zhao, D. Wang, Y. Liu, J. Wang and K. Ying, Phys. Med. Biol., 2019, 64, 195007.
A. Saberi, D. Shahbazi-Gahrouei, M. Abbasian, M. Fesharaki, A. Baharlouei and Z. Arab-Bafrani, Int. J. Radiat. Biol., 2017, 93, 315-323.
B. Wu, S. T. Lu, H. Yu, R. F. Liao, H. Li, B. V. Lucie Zafitatsimo, Y. S. Li, Y. Zhang, X. L. Zhu, H. G. Liu, H. B. Xu, S. W. Huang and Z. Cheng, Biomaterials, 2018, 159, 37-47.
C.-J. Liu, C.-H. Wang, S.-T. Chen, H.-H. Chen, W.-H. Leng, C.-C. Chien, C.-L. Wang, I. M. Kempson, Y. Hwu and T.-C. Lai, Phys. Med. Biol., 2010, 55, 931.
D. B. Chithrani, S. Jelveh, F. Jalali, M. van Prooijen, C. Allen, R. G. Bristow, R. P. Hill and D. A. Jaffray, Radiat. Res., 2010, 173, 719-728.
D. Kim and I. Wang, Med. Phys., 2012, 39, 3872.
D. R. Luffer and K. H. Schram, Rapid Communications in Mass Spectrometry, 1990, 4, 552-556.
G. Cheng, J. Zielonka, D. McAllister, M. Hardy, O. Ouari, J. Joseph, M. B. Dwinell and B. Kalyanaraman, Cancer Lett., 2015, 365, 96-106.
G. Song, L. Cheng, Y. Chao, K. Yang and Z. Liu, Adv. Mater., 2017, 29, 1700996.
H. Chen, G. D. Wang, W. Tang, T. Todd, Z. Zhen, C. Tsang, K. Hekmatyar, T. Cowger, R. Hubbard, W. Zhang, J. Stickney, B. Shen and J. Xie, Adv. Mater., 2014, 26, 6761-6766.
H. Chen, G. D. Wang, X. Sun, T. Todd, F. Zhang, J. Xie and B. Shen, Adv. Funct. Mater., 2016, 26, 3973-3982.
H. Chen, Y. Qiu, D. Ding, H. Lin, W. Sun, G. D. Wang, W. Huang, W. Zhang, D. Lee, G. Liu, J. Xie and X. Chen, Adv. Mater., 2018, 30, 1802748.
J. D. Bradley, R. Paulus, R. Komaki, G. Masters, G. Blumenschein, S. Schild, J. Bogart, C. Hu, K. Forster, A. Magliocco, V. Kavadi, Y. I. Garces, S. Narayan, P. Iyengar, C. Robinson, R. B. Wynn, C. Koprowski, J. Meng, J. Beitler, R. Gaur, W. Curran, Jr. and H. Choy, Lancet Oncol., 2015, 16, 187-199.
J. Deng, S. Xu, W. Hu, X. Xun, L. Zheng and M. Su, Biomaterials, 2018, 154, 24-33.
J. F. Hainfeld, F. A. Dilmanian, Z. Zhong, D. N. Slatkin, J. A. Kalef-Ezra and H. M. Smilowitz, Phys. Med. Biol., 2010, 55, 3045-3059.
J. Kolosnjaj-Tabi, Y. Javed, L. Lartigue, J. Volatron, D. Elgrabli, I. Marangon, G. Pugliese, B. Caron, A. Figuerola, N. Luciani, T. Pellegrino, D. Alloyeau and F. Gazeau, ACS Nano, 2015, 9, 7925-7939.
L. Sun, D. Hu, Z. Zhang and X. Deng, Int. J. Environ. Res. Public Health, 2019, 16, 4773.
M. L. Schipper, N. Nakayama-Ratchford, C. R. Davis, N. W. Kam, P. Chu, Z. Liu, X. Sun, H. Dai and S. S. Gambhir, Nat. Nanotechnol., 2008, 3, 216-221.
M. Zhang, Z. Cui, R. Song, B. Lv, Z. Tang, X. Meng, X. Chen, X. Zheng, J. Zhang, Z. Yao and W. Bu, Biomaterials, 2018, 155, 135-144.
O. Changizi, S. Khoei, A. Mahdavian, S. Shirvalilou, S. R. Mahdavi and J. K. Rad, Photodiagn. Photodyn. Ther., 2020, 29, 101602.
P. Ncube, N. Bingwa, H. Baloyi and R. Meijboom, Appl. Catal., A, 2015, 495, 63-71.
P. Retif, S. Pinel, M. Toussaint, C. Frochot, R. Chouikrat, T. Bastogne and M. Barberi-Heyob, Theranostics, 2015, 5, 1030-1044.
R. Javaid and U. Y. Qazi, Int. J. Environ. Res. Public Health, 2019, 16, 2066.
S. Baker, M. Dahele, F. J. Lagerwaard and S. Senan, Radiat. Oncol., 2016, 11, 115.
S. Bonvalot, C. Le Pechoux, T. De Baere, G. Kantor, X. Buy, E. Stoeckle, P. Terrier, P. Sargos, J. M. Coindre, N. Lassau, R. Ait Sarkouh, M. Dimitriu, E. Borghi, L. Levy, E. Deutsch and J. C. Soria, Clin. Cancer Res., 2017, 23, 908-917.
S. Bonvalot, P. L. Rutkowski, J. Thariat, S. Carrere, A. Ducassou, M. P. Sunyach, P. Agoston, A. Hong, A. Mervoyer, M. Rastrelli, V. Moreno, R. K. Li, B. Tiangco, A. C. Herraez, A. Gronchi, L. Mangel, T. Sy-Ortin, P. Hohenberger, T. de Baere, A. Le Cesne, S. Helfre, E. Saada-Bouzid, A. Borkowska, R. Anghel, A. Co, M. Gebhart, G. Kantor, A. Montero, H. H. Loong, R. Verges, L. Lapeire, S. Dema, G. Kacso, L. Austen, L. Moureau-Zabotto, V. Servois, E. Wardelmann, P. Terrier, A. J. Lazar, J. Bovee, C. Le Pechoux and Z. Papai, Lancet Oncol., 2019, 20, 1148-1159.
S. C. O'Brien, J. R. Heath, R. F. Curl and R. E. Smalley, J. Chem. Phys., 1988, 88, 220-230.
S. E. Page, W. A. Arnold and K. McNeill, J. Environ. Monit., 2010, 12, 1658-1665.
S. Li, I V. Timoshkin, M. Maclean, S. J. Macgregor, M. P. Wilson, M. J. Given, T. Wang and J. G. Anderson, IEEE Trans. Dielectr. Electr. Insul., 2015, 22, 1856-1865.
S. Marrache and S. Dhar, Proc. Natl. Acad Sci. U.S.A., 2012, 109, 16288-16293.
T. J. Mason, J. P. Lorimer, D. M. Bates and Y. Zhao, Ultrason. Sonochem., 1994, 1, S91-S95.
W. N. Rahman, N. Bishara, T. Ackerly, C. F. He, P. Jackson, C. Wong, R. Davidson and M. Geso, Nanomed Nanotechnol. Biol. Med, 2009, 5, 136-142.
X. He, Q. Luo, J. Zhang, P. Chen, H. J. Wang, K. Luo and X. Q. Yu, Nanoscale, 2019, 11, 12973-12982.
X. Ragàs, A. Jimenez-Banzo, D. Sanchez-Garcia, X. Batllori and S. Nonell, Chem. Commun., 2009, 20, 2920-2922.
Y. Hao, Y. Altundal, M. Moreau, E. Sajo, R. Kumar and W. Ngwa, Phys. Med Biol., 2015, 60, 7035-7043.
Y. Li, K. H. Yun, H. Lee, S. H. Goh, Y. G. Suh and Y. Choi, Biomaterials, 2019, 197, 12-19.
Y. Xu, X. H. Jia, X. B. Yin, X. W. He and Y. K. Zhang, Anal. Chem., 2014, 86, 12122-12129.
Y. Yong, X. Cheng, T. Bao, M. Zu, L. Yan, W. Yin, C. Ge, D. Wang, Z. Gu and Y. Zhao, ACS Nano, 2015, 9, 12451-12463.
Y. Zang, L. Gong, L. Mei, Z. Gu and Q. Wang, ACS Appl. Mater. Interfaces, 2019, 11, 18942-18952.

1. A method of making a subject with a cancer more susceptible to radiotherapy or increasing the efficacy of radiation treatment of a cancer in a subject or treating a subject with a cancer or increasing hydroxyl radical production in a tumor in subject undergoing x-ray irradiation, the method comprising administering to the subject gadolinium-intercalated carbon dots.

2. (canceled)

3. (canceled)

4. The method of increasing hydroxyl radical production in a tumor in subject undergoing x-ray irradiation of claim 1, further comprising administering to the subject radiotherapy.

5. The method of claim 1, wherein the gadolinium-intercalated carbon dots further comprise a carboxylic acid or amino group.

6. The method of claim 1, wherein the gadolinium-intercalated carbon dots further comprise a detectable label.

7. The method of claim 1, wherein the gadolinium-intercalated carbon dots are administered before the administration of radiotherapy.

8. The method of claim 1, wherein the gadolinium-intercalated carbon dots are administered concurrent with the administration of radiotherapy.

9. The method of claim 1, wherein the gadolinium-intercalated carbon dots are administered after the administration of radiotherapy

10. The method of claim 1, wherein the gadolinium-intercalated carbon dots comprise nanoparticles of gadolinium encapsulated in an amorphous shell and a mesoporous silica nanoparticle.

11. The method of claim 10, wherein the mesoporous silica nanoparticle has an average diameter of from about 100 nm to about 200 nm.

12. The method of claim 10, wherein the mesoporous silica nanoparticle has an average pore size of from about 1 nm to about 20 nm.

13. The method of claim 10, wherein the mesoporous silica nanoparticle has an average pore size of about 3, about 7, or about 11 nm.

14. The method of claim 10, wherein the nanoparticles of gadolinium have an average diameter of from about 1 nm to about 20 nm.

15. The method of claim 1, wherein the cancer comprises non-small cell lung cancer.
